(19) United States
(12) Patent Application Publication
(10) Pub. No.: US 2016/0187600 A1
(43) Pub. Date: Jun. 30, 2016

(54) SYSTEMS AND METHODS FOR A LIGHT SOURCE BASED ON DIRECT COUPLING FROM LEDS

(71) Applicant: Boston Scientific Scimed, Inc., Maple Grove, MN (US)

(72) Inventors: Kirsten VIERING, Watertown, PA (US); Russell DRESHER, Hudson, MA (US); Paul AQUILINO, Walpole, MA (US); Ryan FOSS, Gray, ME (US)

(73) Assignee: Boston Scientific Scimed, Inc., Maple Grove, MN (US)

(21) Appl. No.: 14/980,136

(22) Filed: Dec. 28, 2015

Related U.S. Application Data
(60) Provisional application No. 62/097,469, filed on Dec. 29, 2014.

(51) Int. Cl. G02B 6/42; A61B 1/07; A61B 1/06; G02B 6/04

(52) U.S. Cl. CPC G02B 6/4292; G02B 6/04; A61B 1/07; A61B 1/0684

(57) ABSTRACT

The present disclosure is directed to a light source. More particularly, aspects of the disclosure relate to coupling light into an optical fiber from a light emitting diode (“LED”). The systems and methods described herein may be utilized in a medical device. More specifically, the light coupled into an optical fiber from an LED located at a proximal end of a device may be emitted from a distal end of an endoscope.

### CROSS-REFERENCE TO RELATED APPLICATIONS

This application claims the benefit of U.S. Provisional Application No. 62/097,469, filed Dec. 29, 2014, the disclosure of which is incorporated herein by reference in its entirety.

### TECHNICAL FIELD

Examples of the present disclosure relate generally to a light source. More particularly, aspects of the disclosure relate to coupling light into an optical fiber from a light emitting diode (“LED”). Aspects of the disclosure also cover methods of using such devices having such coupling.

### BACKGROUND OF THE DISCLOSURE

Endoscopic visualization is used to diagnose and/or treat a variety of conditions in the gastric, pulmonary, and urologic tracts. Endoscopes are not only required to navigate to the target site, but also to provide adequate visualization for diagnosis and/or treatment. In order to provide visualization, light is typically coupled from a light source external to the endoscope into optical fibers housed within the endoscope, which then deliver light to the distal end of the endoscope.

Standard light sources include xenon and halogen lamps. These standard light sources are typically used because they produce relatively bright light. This allows for the coupling of a sufficient amount of light into the optical fibers of the endoscope. Unfortunately, these light sources produce significant amounts of heat and therefore require a cooling mechanism. The addition of a cooling mechanism makes the light source large and expensive. As a result, a dedicated light source unit which is not incorporated into the capital equipment used for image processing may be required. In addition, these light sources are mostly limited to two states, on or off, as electronic control at lower light levels is difficult. Mechanical shutters may be used to adjust the amount of light coupled from the light source to the endoscope, adding to the size and expense of the device.

### SUMMARY OF THE DISCLOSURE

Aspects of the present disclosure provide systems and methods for a compact, low heat, adjustable light source created by coupling light into an optical fiber from an LED.

Additional objects and advantages of the claimed invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

In one example, a light emitting device may include a first section including at least one optical fiber having a light receiving surface proximate a proximal end of the optical fiber, and a second section including a light emitting diode positioned to emit light toward the light receiving surface, wherein first section is attachable to and detachable from the second section.

Examples of the light emitting device may additionally and/or alternatively include one or more other features. For example, the light emitting diode is a surface light emitting diode. A cross-sectional area of the light receiving surface of the at least one optical fiber is larger than a surface area of a light emitting surface of the light emitting diode. A window may be disposed between the at least one optical fiber and the light emitting diode. The at least one optical fiber may be a fiber bundle. The at least one optical fiber may be a single fiber. The at least one optical fiber has a numerical aperture of at least approximately 0.5. At least a portion of the first section is an endoscope. The at least one optical fiber emits light from a distal end of the first section. An adjustment to a current driving the light emitting diode adjusts the brightness of the light emitted from the distal end of the first section. A distance between the light receiving surface of the at least one optical fiber and the light emitting diode is in the range of approximately 0.025 millimeters to approximately 1.015 millimeters. A space between the at least one optical fiber and the light emitting diode is free of a light altering component. A cross-sectional area of the light receiving surface of the at least one optical fiber is smaller than a surface area of a light emitting surface of the light emitting diode. A space between the at least one optical fiber and the light emitting diode may consist of a gas.

In another example, a method providing light may include attaching a medical device to a light source, wherein the medical device includes at least one optical fiber, and the light source includes at least one light emitting diode, wherein a light receiving surface of the at least one optical fiber aligns with a light emitting surface of the light emitting diode; supplying current to the light emitting diode so that light emits from a distal end of the medical device; and detaching the medical device from the light source.

Examples of the method may additionally and/or alternatively include one or more other features. For example, the at least one optical fiber extends from a proximal end of the medical device to a distal end of the medical device. A space between the at least one optical fiber and the light emitting diode is free of a light altering component. The method may include adjusting a current driving the light emitting diode. The medical device is an endoscope. A cross-sectional area of the light receiving surface of the at least one optical fiber is smaller than a surface area of a light emitting surface of the light emitting diode. The at least one optical fiber has a numerical aperture of at least approximately 0.5. After detaching the medical device from the light source, attaching a second medical device to the light source.

In one example, a light emitting device may include a medical device including at least one optical fiber that extends from a proximal end of the medical device to a distal end of the medical device; and a light source may include a light emitting diode, wherein the proximal end of the medical device may be attachable to and detachable from the light source, and a light receiving surface of the at least one optical fiber may align with a hot spot of a light emitting surface of the light emitting diode.

### BRIEF DESCRIPTION OF THE DRAWINGS

The accompanying drawings, which are incorporated in and constitute a part of this specification, illustrate various exemplary aspects and together with the description, serve to explain the principles of the disclosed examples.

FIG. 1 illustrates an example of an endoscopic device, including a direct coupling scheme, an endoscope, and a capital box;

FIG. 2 illustrates an example of a direct coupling scheme, including a proximal end of an optical fiber and an LED;

FIG. 3 illustrates an alternative example of a top view of a direct coupling scheme;

FIG. 4 illustrates an alternative example of a top view of a direct coupling scheme; and

FIG. 5 illustrates an example of a direct coupling scheme including representations of the optical fiber's acceptance cone and a light emittance pattern of the LED.

### DETAILED DESCRIPTION

Reference will now be made in detail to examples of the disclosure, examples of which are illustrated in the accompanying drawings. Wherever possible, the same reference numbers will be used throughout the drawings to refer to the same or like parts. As used herein, the terms “about,” “substantially,” and “approximately,” may indicate a range of values within +/−5% of a stated value.

Aspects of the present disclosure relate to systems and methods for directly coupling light into an optical fiber from an LED. The systems and methods described herein may be utilized in a medical device. More specifically, the light coupled into an optical fiber from an LED located at a proximal end of a device may be emitted from a distal end of an endoscope. Unlike conventional light sources, the present light source may be compact, inexpensive, and disposable. In some implementations, the light source may include having an adjustable brightness, emitting low heat, etc.

In some examples of a direct coupling scheme of the present disclosure, light may be coupled into an optical fiber or fiber bundle located within the endoscope by placing the proximal end of an optical fiber or fiber bundle in close proximity to a light emitting surface of an LED. In some examples, the LED may be located within separate capital equipment. This may result in a low-cost coupling scheme, as no expensive optical elements are required to achieve adequate light coupling. In some examples, the LED may be located in the endoscope and thus the direct coupling may occur within the endoscope itself.

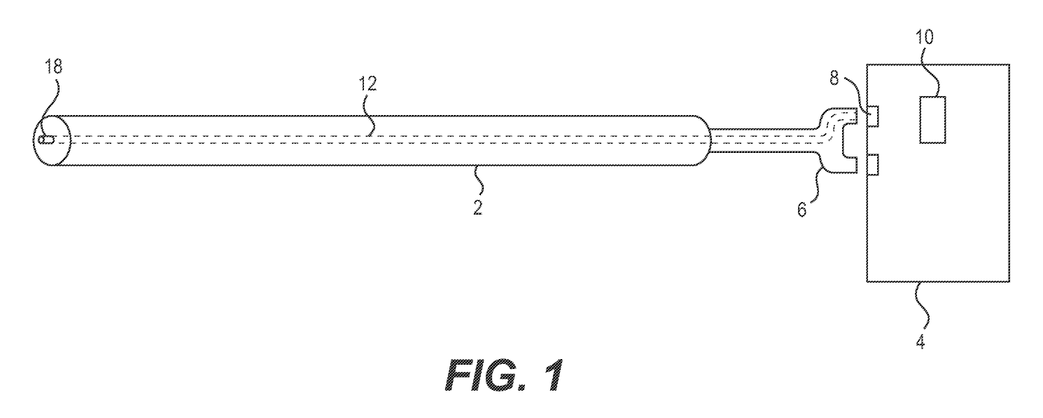

For example, FIG. 1 illustrates such a light source as utilized for an endoscopic device. LED 10 may be part of capital equipment, for example, located in capital box 4. Socket 8 may be situated on a surface of capital box 4, allowing access to the interior of capital box 4 and LED 10. Endoscope 2 may include one or more optical fibers and/or fiber bundles 12. As in conventional endoscopes, endoscope 2 may include imaging and may have any number of lumens for delivery of medical tools, irrigation, suction, etc. The distal end 18 of optical fiber(s) 12 may extend to the distal end of the endoscope 2 through the proximal end of the endoscope 2. In some examples, the distal end 18 of the optical fiber 12 may be flush with the distal end of the endoscope 2. In some examples, the distal end 18 of the optical fiber 12 may protrude from or be recessed within the distal end of the endoscope 2. The proximal end of the endoscope 2 may be attached to or otherwise include a connector 6, and optical fiber(s) 12 may extend the full length of connector 6. Connector 6 may be configured to fit within socket 8. Connector 6 may be selectively attached to or disattached from socket 8 by a user. In other words, a user may connect connector 6 and socket 8 for use, and thereafter disconnect connector 6 from socket 8. This connection may be configured so as to align the optical fiber(s) 12 within the connector 6 with the LED 10 disposed within capital box 4. As shown in FIG. 1, connector 6 includes two prongs. In some examples, both prongs include optical fibers. A second LED may be positioned proximal to the second prong in a manner similar to LED 10 and the first prong. In some examples, other tools or instruments may be disposed within the second prong. For example, electrical current may be carried from capital box 4 through the second prong of connector 6 to the distal end of endoscope 2. In some examples, connector 6 has one prong. In some examples, the element connecting endoscope 2 and capital box 4 may not be a “prong” but may be any structure providing fiber support and/or fiber alignment.

The configuration illustrated in FIG. 1 and the use of this light source within an endoscopic device is merely exemplary. This light source may be used independent of another device or in conjunction with any device in need of a light source.

By supplying power/current to the LED 10 so that it emits light, the light emitted from the LED 10 may be coupled to the optical fiber 12. The coupled light may then be emitted out the distal end 18 of optical fiber 12. The amount of light emitted from LED 10 can be continuously varied by adjusting the current driving the LED 10. It is therefore possible to adjust the light coupled into the fibers 12 by controlling the current through the LED 10 and thus the light emitted from the distal end 18 of the optical fiber 12. This may eliminate the need for a mechanical shutter, and thus reduce the size and cost, as well as lowering the possibility of a mechanical failure of the device. The LED may be supplied by any power source. The power source may be configured to provide the maximum current consumable by the LED. In some examples, the LED 10 may be configured to consume approximately 5 amps.

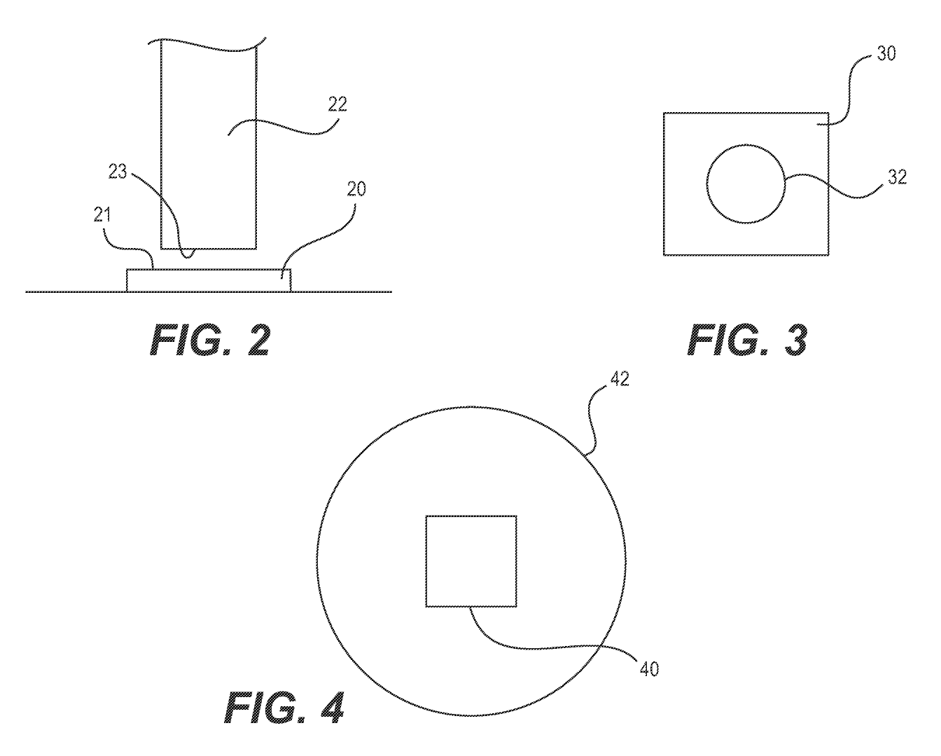

FIG. 2 illustrates a side view of a light receiving surface 23 of optical fiber 22 facing a light emitting surface 21 of LED 20. The optical fiber(s) 22 may be aligned at or in close proximity to the brightest spot (“hot spot”) of LED 20. The location of the “hot spot” may depend on the design of LED 20. For example, if the current is provided to LED 20 from one side only, the “hot spot” may be biased slightly towards that side. If the current is provided in a more symmetrical fashion, however, the “hot spot” may be located near the center of the LED 20.

The acceptable margin of error for aligning optical fiber(s) 22 with the “hot spot” of LED 20 may depend on the exact LED, optical fiber, and/or configuration. In some examples, the acceptable margin of error may be between approximately 0.1 mm and approximately 1.0 mm and preferably approximately 0.025 mm.

The distance between the LED 20 and the light receiving surface 23 of optical fiber 22 may be controlled, as the amount of light coupled decreases significantly as a function of distance to the LED's hot spot, i.e., less light couples to the optical fiber 22 as the distance increases. For example, in some examples, the nominal distance between the optical fiber(s) 12 and the LED 20 may be approximately 0.485 mm, though distances lesser or greater than 0.485 mm may be suitable. For example, the nominal distance may be between approximately 0.1 mm and approximately 1.0 mm. In some examples, the distance and alignment may be controlled by the connector/socket configuration illustrated in FIG. 1.

In some examples, LED 20 and optical fiber 22 may not be in physical contact with each other. LED 20 and optical fiber 22 may be moveable relative to each other. For example, only air may be disposed between the two components. By leaving air in the space between the light receiving surface 23 of optical fiber 22 and the light emitting surface 21 of LED 20 instead of an index matching gel or adhesive, allows a user to more easily separate the LED 20 and optical fiber 22 and replace the optical fiber with a second fiber without having to replace/rework the index matching gel. By allowing for separation of the two, the optical fiber 22 and thus a device containing the optical fiber 22 may be disposable/interchangeable. In some examples, the device containing the optical fiber 22 is an endoscope, like endoscope 2 illustrated in FIG. 1. Additionally, a window may be placed distal to the light emitting surface 21 of LED 20. In some examples, LED 20 may be manufactured with a window, and thus no window need be added. In some examples, this window may be made of glass. Due in part to the adjustable brightness of the LED, no light altering/adjusting components (e.g., mechanical shutters) need be disposed between the LED 20 and the light receiving surface 23 of the optical fiber 22.

In some examples, if optical fiber 22 is a plastic optical fiber, contact between the plastic optical fiber and LED 20 should be avoided, especially when the LED 20 is emitting large amounts of light. The increased temperature at the light emitting surface 21 of the LED 20 may lead to melting of plastic optical fibers onto the LED 20 or the window in front of the LED. This may reduce the amount of light that can be coupled into any fiber that is subsequently placed in front of the LED 20, even if the melted plastic fiber is removed. A window placed between the light receiving surface 23 of optical fiber 22 and the light emitting surface 21 of LED 20 may reduce the risk of contact between the plastic optical fiber and LED 20. Even with the addition of the window, however, melting of a plastic fiber may also lead to damage to LED 20.

As shown in FIGS. 1 and 2, the light source according to some examples may be more compact than traditional light sources for several reasons. Optical fiber 22 is placed directly in front of the LED 20 without the need for coupling optics. Also, an LED light source is typically smaller than xenon or halogen light sources. Further, LEDs traditionally have better conversion efficiency from electrical power to light emittance. This means that LED light sources produce less heat as compared to xenon or halogen bulbs, allowing for a decrease in size of a cooling mechanism. The ability to adjust the brightness of the LED by controlling the current to the LED may mean a mechanical shutter is not necessary, thus also decreasing the size of the light source and its associated equipment.

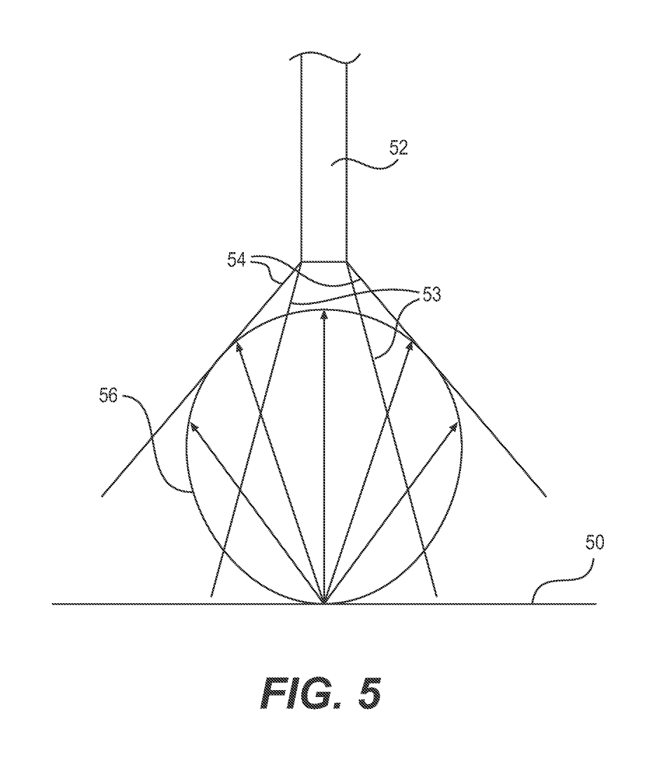

FIGS. 3 and 4 are top views illustrating alternative examples of the LED and optical fibers. FIG. 3 illustrates a top view in which the light emitting surface of LED 30 has a larger surface area than a cross-sectional area of optical fiber 32. The brightness of the LED 30 may depend on the size of the LED 30, thus the larger the LED 30 the more light it may emit. In some examples, based on the LED, it may be advantageous to bias the position of the fiber towards one side of the LED to maximize the amount of light coupled.

In the example illustrated in FIG. 4, the light emitting surface of LED 40 has a smaller surface area than the cross-sectional area of optical fiber 42. The coupling efficiency may be increased by increasing the diameter of the optical fiber so that it is larger than the size of the LED. The relative size of the LED and the optical fiber each depends, in part, on the particular application and/or device the light source is being used in conjunction with. For example, the diameter of the optical fiber may be limited by the diameter of the endoscope it is designed to fit within.

The LED may be any size or shape. The larger the LED, the more light the LED may emit and be subsequently coupled to an optical fiber(s). The LED also may need to be sufficiently small to fit within the desired device. For example, the LED may be approximately 2 mm by 2 mm. This size may fit within the capital box 4 connectable to endoscope 2 as illustrated in FIG. 1. The LED may emit light at any wavelength. For example, the LED may emit light within the visible range and/or specifically white light.

Any type of LED that emits light in a variety of patterns may be used in this direct coupling scheme. In some examples, the LED may be a surface light emitting diode (sLED). An sLED may be utilized in this direct coupling scheme due to its light emittance profile. For example, an sLED may produce more directional light than other LEDs and increased directional light may provide for better coupling. SLEDs may emit light in a Lambertian pattern. FIG. 5 illustrates a representation of light 56 emitted in a Lambertian pattern from LED 50.

The optical fiber(s) illustrated in FIGS. 1-5 may be any number of optical fibers, including a single fiber or a fiber bundle. A single optical fiber may be more efficient at capturing light, because a fiber bundle may include dead space between individual fibers. A fiber bundle may include any number of optical fibers, the fiber bundle and the individual fibers may have any diameter. For example, the larger the diameter, the more light that may be delivered to the distal end of the optical fiber. The diameter may be limited as the optical fiber must be small enough for the desired implementation. For example, the diameter of the optical fiber 12 of may be approximately 500 microns so that the endoscope 2 and/or connector 6 may accommodate the optical fiber 12. The optical fiber(s) may be made of any material, including plastic, glass, and/or a combination of plastic and glass within a bundle.

The maximum coupling efficiency between LED 50 and optical fiber 52 may be achieved, when all of the light emitted by the LED 50 (represented by Lambertian pattern 56) falls within the light acceptance cone (represented by 54) of the optical fiber 52, as shown in FIG. 5. Alternatively, a smaller light acceptance cone (represented by 53) may not achieve maximum coupling efficient as at least some of the light emitted by the LED 50 (represented by Lambertian pattern 56) falls outside the smaller acceptance cone 53. In some examples, coupling efficiency may be increased by increasing the diameter of the optical diameter so that it is larger than the size of the LED, as shown in FIG. 4. Additionally or alternatively, coupling efficiency may be increased as the numerical aperture (NA) of the optical fiber increases. The larger the NA of the fiber, the larger the acceptance cone, meaning light from more directions can be coupled into the fiber. For example, as shown in FIG. 5, the wider the acceptance cone 54, the more of the light emitted 56 from LED 50 may be coupled into optical fiber 52. In some examples, the NA of the optical fiber may be greater than approximately 0.5. In some examples, the NA of the optical fiber may be approximately 0.63.

The many features and advantages of the disclosure are apparent from the detailed specification, and thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and accordingly, all suitable modifications and equivalents may be resorted to, falling within the scope of the disclosure.

Other aspects of the disclosure will be apparent to those skilled in the art from consideration of the specification and practice of the invention disclosed herein. It is intended that the specification and examples be considered as exemplary only, with a true scope and spirit of the disclosure being indicated by the following claims.

What is claimed is:

1. A light emitting device, comprising:
a first section including at least one optical fiber having a light receiving surface proximate a proximal end of the optical fiber; and
a second section including a light emitting diode positioned to emit light toward the light receiving surface, wherein first section is attachable to and detachable from the second section.

2. The light emitting device of claim 1, wherein the light emitting diode is a surface light emitting diode.

3. The light emitting device of claim 1, wherein a cross-sectional area of the light receiving surface of the at least one optical fiber is smaller than a surface area of a light emitting surface of the light emitting diode.

4. The light emitting device of claim 1, wherein a window is disposed between the at least one optical fiber and the light emitting diode.

5. The light emitting device of claim 1, wherein the at least one optical fiber is a fiber bundle.

6. The light emitting device of claim 1, wherein the at least one optical fiber is a single fiber.

7. The light emitting device of claim 1, wherein the at least one optical fiber has a numerical aperture of at least approximately 0.5.

8. The light emitting device of claim 1, wherein at least a portion of the first section is an endoscope.

9. The light emitting device of claim 1, wherein the at least one optical fiber emits light from a distal end of the first section.

10. The light emitting device of claim 9, wherein an adjustment to a current driving the light emitting diode adjusts the brightness of the light emitted from the distal end of the first section.

11. The light emitting device of claim 1, wherein a distance between the light receiving surface of the at least one optical fiber and the light emitting diode is in the range of approximately 0.025 millimeters to approximately 1.015 millimeters.

12. The light emitting device of claim 1, wherein a space between the at least one optical fiber and the light emitting diode is free of a light altering component.

13. A method for providing light, comprising:
attaching a medical device to a light source, wherein the medical device includes at least one optical fiber, and the light source includes a light emitting diode, wherein a light receiving surface of the at least one optical fiber aligns with a light emitting surface of the light emitting diode;
supplying current to the light emitting diode so that light emits from a distal end of the medical device; and
detaching the medical device from the light source.

14. The method of claim 13, wherein the at least one optical fiber extends from a proximal end of the medical device to a distal end of the medical device.

15. The method of claim 13, wherein a space between the at least one optical fiber and the light emitting diode is free of a light altering component.

16. The method of claim 13, furthering comprising:
adjusting a current driving the light emitting diode.

17. The method of claim 13, wherein a cross-sectional area of the light receiving surface of the at least one optical fiber is smaller than a surface area of a light emitting surface of the light emitting diode.

18. The method of claim 13, wherein the medical device is an endoscope.

19. The method of claim 13, further comprising:
after detaching the medical device from the light source, attaching a second medical device to the light source.

20. A light emitting device, comprising:
a medical device including at least one optical fiber extending from a proximal end of the medical device to a distal end of the medical device; and
a light source including a light emitting diode, wherein the proximal end of the medical device is attachable to and detachable from the light source, and a light receiving surface of the at least one optical fiber aligns with a hot spot of a light emitting surface of the light emitting diode.
